(19) United States
(12) Patent Application Publication
(10) Pub. No.: US 2008/0004486 A1
(43) Pub. Date: Jan. 3, 2008

(54) COMPRESSIVE COUPLING OF AN IMPLANTABLE HEARING AID ACTUATOR TO AN AUDITORY COMPONENT

(75) Inventors: Travis Rian Andrews, Loveland, CO (US); James Frank Kasic, Boulder, CO (US); William J. Simms, Louisville, CO (US); Jose H. Bedoya, Boulder, CO (US)

Correspondence Address: MARSH, FISCHMANN & BREYFOGLE LLP, 3151 SOUTH VAUGHN WAY, SUITE 411, AURORA, CO, 80014, US

(73) Assignee: OTOLOGICS, LLC, Boulder, CO (US)

(21) Appl. No.: 11/763,257

(22) Filed: Jun. 14, 2007

Related U.S. Application Data
(60) Provisional application No. 60/804,765, filed on Jun. 14, 2006.

(51) Int. Cl. A61F 11/00

(52) U.S. Cl. 600/25

(57) ABSTRACT

Apparatus and methods are provided for maintaining a desired centered relationship between a vibratory actuator of an implantable hearing aid transducer and an auditory component post-implantation. In certain embodiments, at least two guide members may extend beyond a distal end of a vibratory actuator for positioning on opposing sides of an auditory component. The guide arms may be employed to restrict post-implantation auditory component movement, and additionally or alternatively, to apply a spring-loading force against an auditory component to reposition and thereby center such auditory component in the event of post-implantation auditory component movement. In certain embodiments, a distal end may be provided on a vibratory actuator, wherein the distal end has a plurality of differently-shaped concave surfaces. A selected one of the different concave surfaces may be positioned for contact engagement with an auditory component to optimize surface engagement. In one embodiment, a contact surface may be rotatably and pivotably disposed at the distal end of a vibratory actuator to facilitate positioning of the contact surface at an optimal orientation relative to an auditory component.

### CROSS REFERENCE TO RELATED APPLICATIONS

This application claims priority under 35 U.S.C. 119 to U.S. Provisional Application No. 60/804,765 entitled “COMPRESSIVE COUPLING OF AN IMPLANTABLE HEARING AID ACTUATOR TO AN AUDITORY COMPONENT,” having a filing date of Jun. 14, 2006, the contents of which are incorporated herein as if set forth in full.

### FIELD OF THE INVENTION

The invention is related to the field of hearing aids, and in particular, to the contact interface between an implantable hearing aid transducer and a component of the auditory system.

### BACKGROUND OF THE INVENTION

Implantable hearing aids entail the subcutaneous positioning of some or all of various hearing augmentation componentry on or within a patient's skull, typically at locations proximate the mastoid process. Implantable hearing aids may be generally divided into two classes, semi-implantable and fully implantable. In a semi-implantable hearing aid, components such as a microphone, signal processor, and transmitter may be externally located to receive, process, and inductively transmit a processed audio signal to implanted components such as a receiver and transducer. In a fully implantable hearing aid, typically all of the components, e.g., the microphone, signal processor, and transducer, are located subcutaneously. In either arrangement, a processed audio signal is provided to a transducer to stimulate a component of the auditory system

By way of example, one type of implantable transducer includes an electromechanical transducer having a magnetic coil that drives a vibratory actuator. The actuator is positioned to mechanically stimulate the ossicles via physical contact. (See e.g., U.S. Pat. No. 5,702,342). Generally, such a vibratory actuator is mechanically engaged (i.e., coupled) with the ossicles during mounting and positioning of the transducer within the patient. In one example, such coupling may occur via a small aperture formed in the incus bone that is sized to receive a tip of the electromechanical transducer. In such an arrangement, the transducer tip may expansively contact the sides of the aperture, may be adhered within the aperture or tissue growth (e.g., osteointegration) may couple the transducer tip to the bone. One disadvantage of methods requiring a hole in the ossicle to facilitate attachment is that a surgical laser must be employed to ablate the ossicle's surface. The laser ablation procedure is burdensome and time consuming. Also, the required equipment is expensive and not present in every surgical setting.

In other arrangements, clamps and/or clips are utilized to couple the vibratory actuator to an ossicle. However, such approaches can entail difficult implant procedures and yield sub-optimum coupling.

As will be appreciated, coupling with the ossicles poses numerous challenges. For instance, during positioning of the transducer, it is often difficult for an audiologist or surgeon to determine the extent of the coupling, or in other words, how well the actuator is attached to the ossicles. Additionally, due to the size of the transducer relative to the ossicles, it is difficult to determine if loading exists between the ossicles and transducer. In this regard, precise control of the engagement between the actuator of the transducer and the ossicies is of critical importance as the axial vibrations can only be effectively communicated when an appropriate interface or load condition exists between the transducer and the ossicles. Overloading of the actuator can result in damage or degraded performance of the biological aspect (e.g., movement of the ossicies) as well as degraded performance of the mechanical aspect (e.g., movement of the vibratory member). Additionally, an underloaded condition, i.e., one in which the actuator is not fully connected to the ossicles, may result in reduced performance of the transducer. Further, once coupled for an extended period, the maintenance and/or replacement with a next generation transducer may be difficult. That is, in many coupling arrangements it may be difficult to de-couple a vibratory actuator/transducer.

### SUMMARY OF THE INVENTION

In view of the foregoing, a primary object of the present invention is to simplify and improve implantation procedures for implantable devices, such as hearing aid transducers. Another object of the present invention is to allow for relative movement (e.g., lateral movement) between a component of the auditory system and an electromechanical transducer to account for physical variations of the auditory component caused by, for example, pressure changes, swallowing, etc. Another object is to provide auditory component engagement means that allows for easily disengaging an auditory component.

One or more of the above objectives and additional advantages may be realized utilizing a contact or ‘force loading’ interface between a vibratory actuator of an implantable transducer and a component of the auditory system. In this regard, a distal portion of the vibratory actuator may be pressed against an auditory component, e.g., the ossicles, to provide a predetermined acceptable load on the component. Tissue attached to the auditory component (e.g., ligaments) may maintain the actuator in contact with the auditory component for both positive and negative actuator displacement (e.g., axial displacement during operation of the implantable transducer.) In this regard, it has been determined that it is not necessary to physically attach the transducer tip to the ossicle bone utilizing, for example, a hole drilled into the bone or by using a clip or clamp arrangement that extends around the ossicle bone to mount the transducer tip to the bone. That is, the compressive contact or “force loading” of the ossicle bone provides the necessary contact for stimulation purposes.

In order to maintain the force loading between the vibratory actuator and an auditory component after an implant procedure it has further recognized that it may be desirable to limit lateral movement of the auditory component relative to the actuator and/or realign the auditory component with the actuator after such lateral movement. For instance, an ossicle bone may move laterally (e.g., in a direction transverse to a vibratory direction of the actuator) as a result of pressure changes (e.g., changes in altitude) and/or physical movements of the patient (e.g., yawning). For purposes hereof, any such movement may be referred to as post-implantation auditory component movement.

In this regard, it has been determined that use of a force loading system may be facilitated by use of a centering device that works to realign the actuator and force loaded auditory component and/or limit relative movement therebetween post-implantation. That is, inventive centering apparatus are provided that may be interconnected or interconnectable to an implantable hearing aid transducer to maintain a desired contact relationship between a vibratory actuator of the transducer and an auditory component post-implantation.

In one aspect, a centering device may include at least two guide members that may extend in a direction defined by at least a distal end portion of the vibratory actuator that engages an auditory component, wherein the guide members function to laterally align a vibratory actuator and an auditory component in the noted direction. In one feature, each of the guide members, as interconnected to an implantable hearing aid transducer, may extend beyond a distal end of a vibratory actuator, wherein the guide members may be positioned on opposing sides of an auditory component. In another feature, each of the guide members may comprise a compliant portion that is deflectable away from a contact axis (e.g., a center axis extending through a distal end surface of the vibratory actuator that contacts an auditory component for communicating vibrations thereto), wherein the guide member(s) may apply a spring force against an auditory component in response to post-implantation auditory component movement and auditory component repositioning may be realized. In yet another feature, each of the two guide members may include a portion that extends away from the contact axis to facilitate initial positioning relative to an auditory component, without clamping the auditory component.

In one approach, the centering device may include opposing first and second compliant (e.g., spring-loaded) guide wires that are interconnected to a support member. Such guidewires may be bent (e.g., plastically deformed) for initial contact positioning on opposing sides of an auditory component, and may further be deflectable from a bent configuration to apply lateral spring forces to an auditory component in response to post-implantation auditory component movement. In another approach, the centering device may include compliant opposing first and second armatures interconnected to a support member. In this regard, distal ends of each of the first and second armatures may extend outwardly away from one another.

In either approach the support member may be affixed to a moving or non-moving portion of an implantable transducer (e.g., a portion that moves upon transducer operation or a portion that is stationary upon transducer operation). The guide wires and armatures of the two noted approaches may function to limit the lateral movement of the auditory component (e.g., depending on their stiffness) and/or to apply a lateral force to the auditory component in response to lateral movement of the auditory component so as to re-center an auditory component with a vibratory actuator.

In another approach, a centering device may comprise at least two guide members that are one of interconnected and interconnectable to a distal end of the vibratory actuator. In this regard, the guide members may be defined by a distal tip having at least two opposing surface portions that define an included angle therebetween (e.g., for receiving an auditory component during use). In turn, either or both of the surface portions may contact an auditory component upon post-implantation auditory component movement to limit relative movement between an auditory component and a distal end of a vibratory actuator. In one embodiment, the at least two surface portions may be integrally defined by a distal tip (e.g., having a U-shaped or V-shaped configuration.)

According to another aspect, a contact surface of a distal tip of a vibratory actuator may be formed to have first and second different concave surfaces. Such concave surfaces may have first and second curvatures.

For example, the first and second concave surfaces may each be partial conical surfaces whose corresponding center axes intersect a center axis of the distal tip at different angles and/or whose radii of curvature are different. In one arrangement, the contact surface may include first and second wings. In turn, first and second concave surfaces may extend between the wings and thereby form a ‘saddle’ configuration to receive an auditory component. Advantageously, during implantation the first concave surface or the second concave surface may be selectively positioned for contact with an auditory component (e.g., so as to increase the area of contact).

In a further aspect, a centering device may comprise at least one contact surface that is rotatably and/or pivotably disposed at a distal end of a vibratory actuator. In turn, the contact surface may be rotated and/or pivoted relative to the distal end of a vibratory actuator so as to achieve optimal contact positioning of the contact surface with an auditory component.

In one aspect, two opposing contact surfaces may be provided that define an opening therebetween (e.g., for receipt of an auditory component), wherein the opening orientation may be selectively adjusted in at least one dimension relative to a contact axis (e.g., a center axis of a distal end portion of a vibratory actuator). In certain embodiments, an opening may be provided that is selectively adjustable in two dimensions relative to a contact axis. For example, a concave surface may be defined by a tip that is rotatably and pivotably interconnected to a distal end of a vibratory actuator.

In one arrangement, a contact surface is formed on a connecting tip that is adapted to fit over a portion of the actuator. In such an arrangement, the connector tip may include an aperture for receiving a tip of the actuator.

An inventive method is also provided for use of the connection with mechanical stimulation of an auditory component by an implantable hearing aid transducer. The method includes the step of contacting a distal end of a vibratory actuator of an implantable hearing aid transducer with an auditory component, wherein the vibratory actuator is displaceable in response to operation of the implantable hearing aid transducer. The method further includes the step of maintaining a desired centered relationship between the distal end of the vibratory actuator and the auditory component post-implantation.

The method may further include a step of positioning at least two guide members on opposing sides of an auditory component, wherein the two guide members may be supportably interconnected to the implantable hearing aid transducer. In turn, the maintaining step may comprise at least one of engaging at least one of the guide members with a lateral aspect of an auditory component to restrict post-implantation auditory component movement, and applying a spring-loaded force by at least one of the two guide members against an auditory component (e.g., a lateral aspect thereof) in response to post-implantation auditory component movement (e.g., so as to reposition the auditory component to a desired centered position relative to a distal end of the vibratory actuator).

In another aspect, the contacting step may include the step of selecting one of a plurality of the differently-shaped contact surfaces provided at a distal end of a vibratory actuator for contact with an auditory component. In this regard, each of the plurality of differently-shaped concave surfaces may present partial conical surfaces whose corresponding center axis intersect a center axis of a distal end of a vibratory actuator (e.g., a contact axis) at different angles and/or whose radii of curvature are different.

In yet another aspect, the method may include a step of positioning at least one contact surface, supportably disposed at a distal end of a vibratory actuator, by selectively adjusting the orientation of the contact surface in at least one dimension relative to the vibratory actuator. For example, such positioning may comprise rotating the contact surface(s) and/or pivoting the contact surface(s) relative to a distal end of a vibratory actuator, then advancing the actuator toward an auditory component to achieve an optimal contact interface. In turn, where two contact surfaces are employed to define an opening therebetween, the opening may be selectively oriented during implantation to yield enhanced post-implantation centering functionality

Additional aspects and advantages will be apparent upon consolidation of the embodiments described below.

### BRIEF DESCRIPTION OF THE DRAWINGS

FIG. 1 illustrates a fully implantable hearing instrument as implanted in a wearer's skull.

FIG. 2 illustrates a force loading connection between a transducer vibratory actuator tip and an auditory component.

FIGS. 3a-3d illustrate one embodiment of a force loading connection apparatus that provides centering for a transducer vibratory actuator tip.

FIG. 4 illustrates the force loading connection apparatus of FIGS. 3a-3d attached to a transducer and engaging an auditory component.

FIG. 5 illustrates the force loading connection apparatus of FIGS. 3a-3d attached to a transducer and engaging an auditory component.

FIG. 6 illustrates another embodiment of a force loading connection apparatus that provides centering for a transducer tip.

FIGS. 7a-7e illustrate one embodiment of a force loading connection tip that attaches over a transducer vibratory actuator tip and provides centering for the transducer vibratory actuator tip.

FIG. 8 illustrates a cross sectional view of the force loading connection tip of FIGS. 7a-e engaging an auditory component.

FIG. 9 illustrates the force loading connection tip of FIGS. 7a-e engaging an auditory component.

FIGS. 10a-10e illustrate another embodiment of a force loading connection tip that attaches over a transducer vibratory actuator tip and provides centering for the transducer vibratory actuator tip.

FIG. 11 illustrates the force loading connection tip of FIG. 9 engaging an auditory component.

FIGS. 12a-12f illustrate another embodiment of a force loading connection tip that attaches over a transducer vibratory actuator tip and provides centering for a transducer vibratory actuator tip.

FIG. 13 illustrates the force loading connection tip of FIG. 12 engaging an auditory component.

FIGS. 14a-b illustrate the force loading tip of FIG. 12 engaging an auditory component.

FIGS. 15a-b illustrate another embodiment of a force loading apparatus that provides centering for a transducer vibratory actuator tip.

FIGS. 16a-16e illustrate a force loading connection tip of the force loading connector apparatus of FIGS. 15a-b.

### DETAILED DESCRIPTION

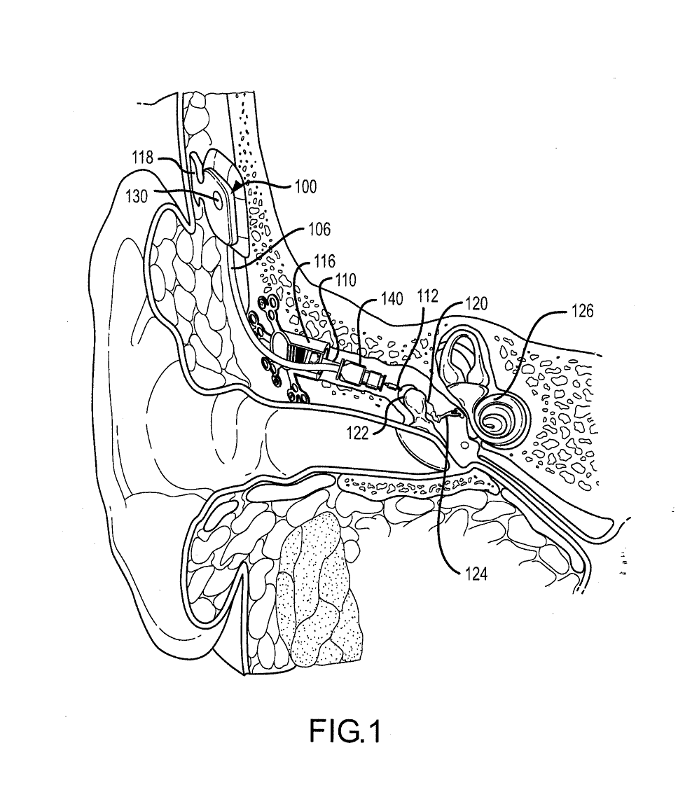

FIG. 1 illustrates one application of the present invention. As illustrated, the application comprises a fully implantable hearing instrument system. As will be appreciated, certain aspects of the present invention may be employed in conjunction with semi-implantable hearing instruments as well and, therefore, the illustrated application is for purposes of illustration and not limitation.

In the illustrated system, a biocompatible implant housing 100 is located subcutaneously on a patient's skull. The implant housing 100 includes a signal receiver 118 (e.g., comprising a coil element) and a microphone 130 that is positioned to receive acoustic signals through overlying tissue. The signal receiver 118 may be utilized for transcutaneously re-charging an energy storage device within the implant housing 100 as well as for receiving program instructions for the hearing instrument system.

The implant housing 100 may be utilized to house a number of components of the fully implantable hearing instrument. For instance, the implant housing 100 may house an energy storage device, a microphone transducer, and a signal processor. Various additional processing logic and/or circuitry components may also be included in the implant housing 100 as a matter of design choice. Typically, the signal processor within the implant housing 100 is electrically interconnected via wire 106 to an electromechanical transducer 140.

The transducer 140 is supportably connected to a positioning system 110, which in turn, is connected to a bone anchor 116 mounted within the patient's mastoid process (e.g., via a hole drilled through the skull). The transducer 140 includes a connection apparatus 112 for connecting the transducer 140 to the ossicies 120 of the patient. In a connected state, the connection apparatus 112 provides a communication path for acoustic stimulation of the ossicles 120, e.g., through transmission of vibrations to the incus 122. As will be more fully discussed herein, the connection apparatus may form a compressive contact interface between the transducer 140 and the ossicles.

The bone anchor 116 may be of a type as taught in U.S. Pat. No. 6,293,903 entitled “APPARATUS AND METHOD FOR MOUNTAING IMPLANTABLE HEARING AID DEVICE”, issued Sep. 25, 2001, the entirety of which is hereby incorporated by reference. Further, the positioning system 110 may be of the type as generally taught by U.S. Pat. No. 6,491,622 entitled “APPARATUS AND METHOD FOR POSITIONING AN IMPLANTABLE HEARING AID DEVICE” issued Dec. 10, 2002, the entirety of which is hereby incorporated by reference.

In short, the positioning system 110 may include a carrier assembly and a swivel assembly that allow for selective three-dimensional positioning of the transducer 140 and connection apparatus 112 at a desired location within a patient. In this regard, the transducer 140 may be supportively connected to a first end of the carrier assembly. In turn, the carrier assembly may be supportively received and selectively secured in an opening defined through a ball member that is captured between plates of the swivel assembly. The interface between the carrier assembly and swivel assembly provides for pivotable, lateral positioning of the first end of the carrier assembly and of the transducer 140 interconnected thereto. That is, the carrier assembly may pivot upon rotation of the ball member, thereby allowing the connection apparatus 112 to be moved along an arcuate path to a desired position. In turn, the interconnected plates may be selectively secured to a bone anchor 116 to maintain a selected pivotal orientation. Further, the carrier assembly may be selectively secured along a continuum positions within the opening of the swivel assembly, thereby facilitating linear advancement/retraction of the carrier assembly and interconnected transducer 140 connection apparatus 112 in a depth dimension. Additionally, the carrier assembly may be defined so that its first end may be selectively advanced and retracted in the depth of dimension relative to an outer support member thereof (e.g., by utilizing a lead screw arrangement), thereby further facilitating selective linear positioning of the transducer 140 and connection apparatus.

As may be appreciated, in relation to an implementation as shown in FIG. 1, the positioning system 110 may be employed to move (e.g., advance or retract) the connection apparatus 112 toward an auditory component by moving the carrier assembly relative to the swivel assembly, by moving the first end of the carrier assembly relative to the support member thereof and/or by pivoting the carrier assembly relative to the plates of the swivel assembly.

During normal operation, acoustic signals are received subcutaneously at the microphone 130. Upon receipt of the acoustic signals, a signal processor within the implant housing 100 processes the signals to provide a processed audio drive signal (e.g., a transducer drive signal) via wire 106 to the transducer 140. As will be appreciated, the signal processor may utilize digital processing techniques to provide frequency shaping, amplification, compression, and other signal conditioning, including conditioning based on patient-specific fitting parameters. The audio drive signal causes the transducer 140 to transmit vibrations at acoustic frequencies to the connection apparatus 112 to effect the desired sound sensation via mechanical stimulation of the incus 122 of the patient. These vibrations are then transmitted from the incus 122 to the stapes 124, effecting a stimulation of the cochlea 126.

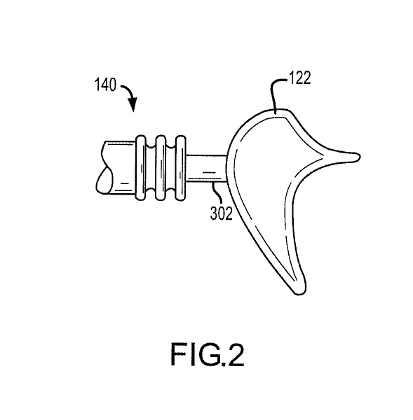

FIG. 2 illustrates a distal end tip 302 of a vibratory actuator of the transducer 140 as disposed proximate to the incus 122. It has been determined that adequate transfer of mechanical energy (e.g., vibrations) from the transducer 140 to the incus 122 may be achieved by a contact/compressive loading of the tip 302 of the vibratory actuator of transducer 140 to the incus 122 or other ossicle bone as the case may be. That is, the transducer 140 may be advanced until the tip 302 contacts the incus 122 and a predetermined force is applied to the incus 122. Ligaments (not shown) are connected to the ossicular chain. These ligaments counteract the force applied by the transducer 140. Stated otherwise, the ligaments pull the incus towards its unloaded or static location and thereby against the tip 302.

When the transducer 140 is operated to displace the vibratory actuator tip 302 (e.g., axially), the transducer tip 302 may be advanced towards the incus 122. Accordingly, the incus 122 is displaced (i.e., to the right as shown in FIG. 2). In contrast, when the transducer tip 302 is retracted relative to the incus 122, the ligaments interconnected to the incus 122 pull the incus back towards its static location as the vibratory actuator tip 302 retracts (i.e., to the left in FIG. 2). As may be appreciated, depending on the initial loading of the incus 122, the incus 122 may be operative to move in contact with the tip 302 for both positive and negative transducer tip displacements relative to a stationary tip location (i.e., zero displacement). In this regard, it has been determined that it is not necessary to physically attach the vibratory actuator tip to the ossicle bone utilizing, for example, a hole drilled into the bone or by using a clip or clamp arrangement that extends around the ossicle bone to mount the tip to the bone. That is, the compressive contact or “force loading” of the ossicle bone provides the necessary contact for stimulation purposes.

One consideration regarding the force-loading concept relates to the tendency of the ossicular chain and/or individual ossicle bones to move after implantation in response to changes in environment. For instance, the incus 122 may move laterally (e.g., perpendicular to the page in FIG. 2) relative to the axis defined by the transducer tip 302. Such movement may be the result of pressure changes (e.g., changes in altitude) affecting the ossicular chain and/or physical movements of the patient (e.g., yawning). In such instances of post-implantation auditory component movement, the lack of the direct mechanical connection between the transducer tip 302 and incus 122 (or other auditory component) in the force loading system may result in the misalignment of the transducer 140 relative to the stimulated auditory component. In this regard, it has been determined that use of a force loading system may be facilitated by use of a centering device that works to realign and/or otherwise maintain alignment of the vibratory actuator tip 302 of the transducer 140 and a force loaded auditory component after implantation.

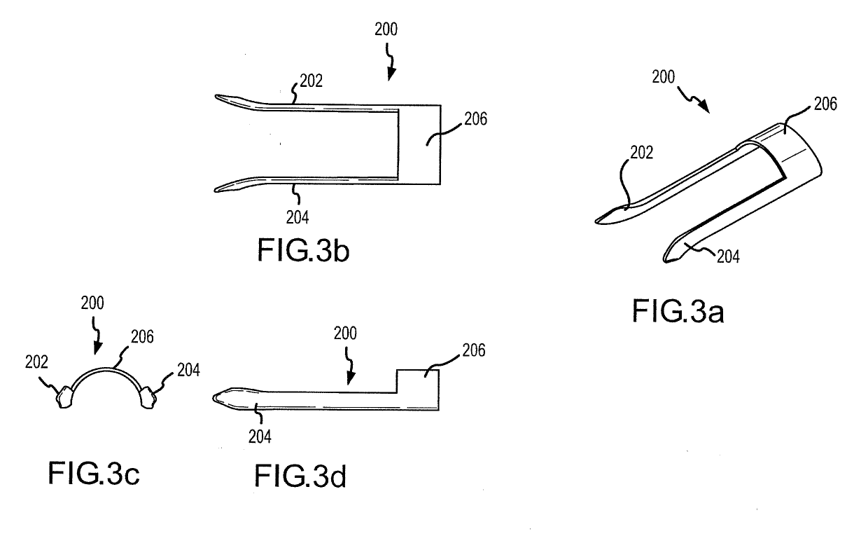

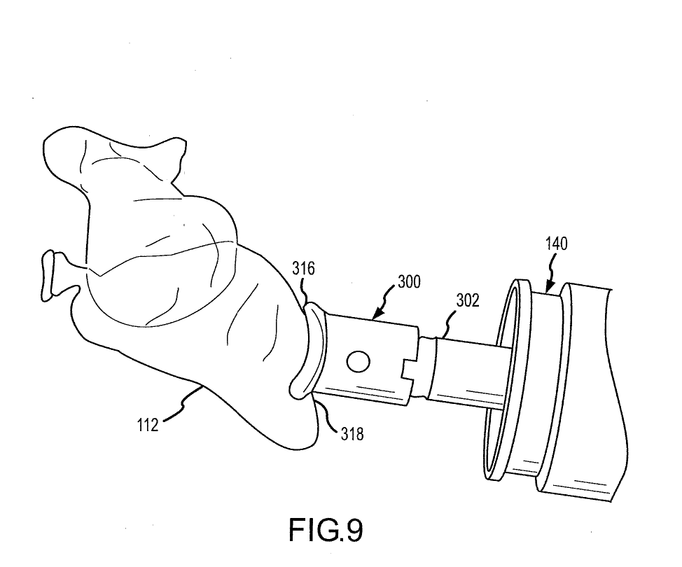

FIGS. 3-16 illustrate numerous embodiments of force loading connection apparatuses that provide centering for a transducer to ossicle interface. However, it is anticipated that numerous other examples of the connection apparatus may be utilized according to the present principles. In addition, those skilled in the art will appreciate how the features described below can be combined to form numerous other examples of the present connection apparatus.

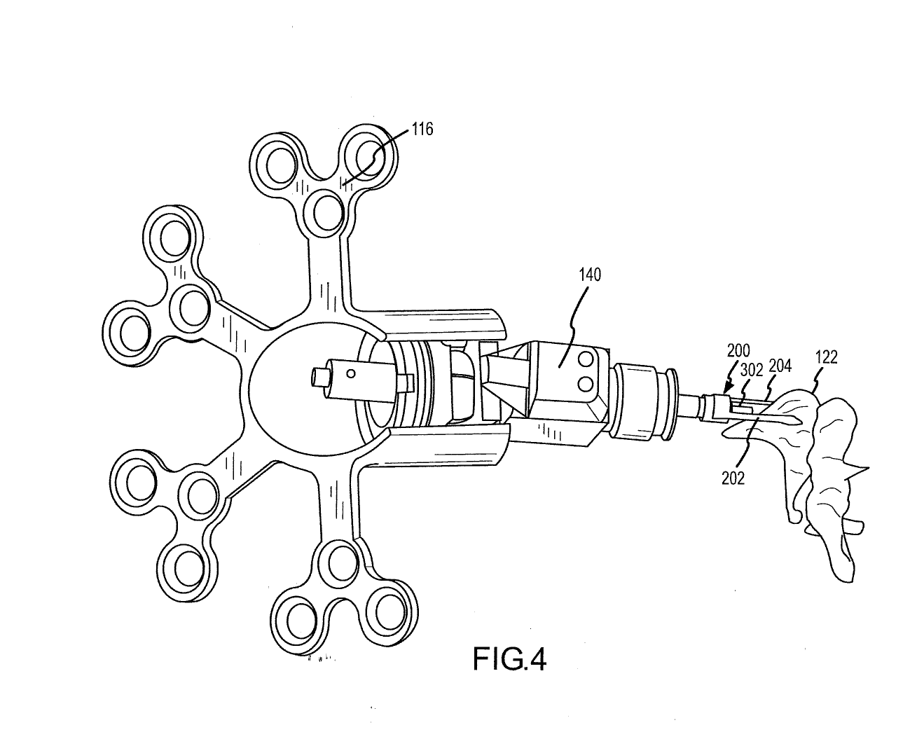

FIGS. 3-5 show a first embodiment of a connection apparatus formed as a guide assembly 200 that may be utilized with a transducer vibratory actuator tip 302 (e.g., see FIG. 4). As shown, the guide assembly 200 includes first and second guide armatures 202, 204 and an interconnecting member 206 that extends between the guide armatures 202, 204. The guide armatures are sized to, when connected to the transducer 140, extend beyond the transducer vibratory actuator tip 302. The connecting member 206 is curved to match the curvature of the outside surface of the vibratory actuator of the transducer 140 for co-movement therewith. In this regard, the guide assembly 200 may be attached to a curved outside surface of a vibratory component of the transducer 140. In another arrangement, the guide assembly 200 is interconnected to a stationary portion of the transducer 140. That is, the guide assembly 200 does not move with movement of the transducer tip 302 the transducer 140.

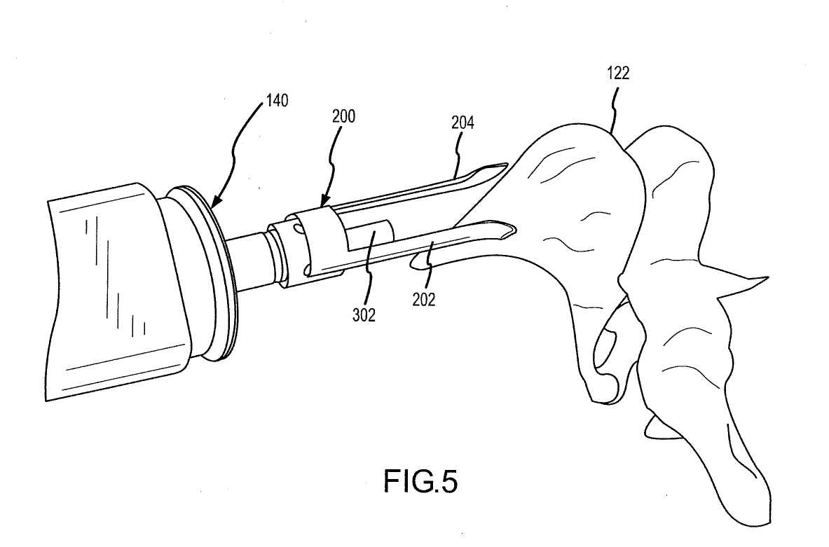

As shown in FIGS. 4 and 5, the first and second guide armatures 202, 204 are spaced such that they may be disposed on opposing surfaces of an ossicle bone such as the incus 122. Referring again to FIG. 3, it is noted that the tips of the armatures 202, 204 turn outward to facilitate engagement of the guide assembly 200 with an ossicle bone. Further, it will be noted that the armatures 202, 204 may elastically deform outward relative to the incus 122 as the transducer 140 is advanced thereto. In this regard, the transducer 140 may be advanced until the tip 302 engages the incus 122 and a predetermined force is applied thereto. Thereafter, the guide assembly 200 may work to counteract the lateral movement of the ossicular chain and/or realign the ossicular chain with the stationary transducer 140 (e.g., by applying a spring-loaded force to the ossicular chain). In the latter regard, it will be noted that the firm skull mounting provided by the bone bracket 116 maintains the transducer 140 in a fixed positional relationship to the skull of the wearer. Accordingly, the guide assembly 200 may apply a lateral force to a lateral aspect the ossicular chain to realign the ossicle bones with the fixed transducer 140 when those bones move laterally to the transducer tip 302 after implantation.

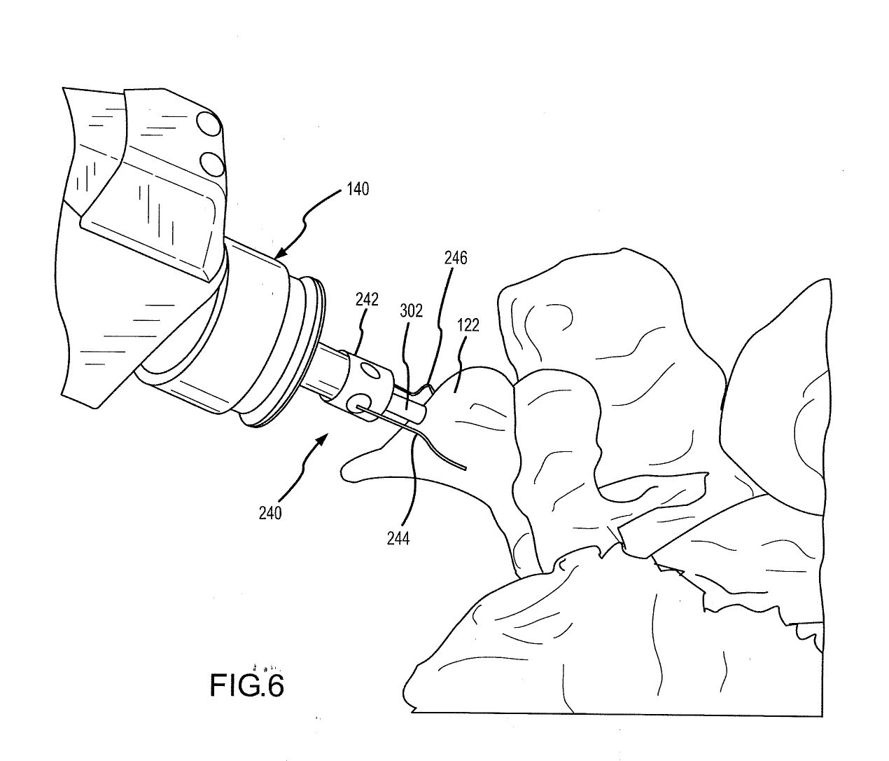

FIG. 6 illustrates a second embodiment of a guide assembly 240 that may be utilized to align a transducer vibratory actuator tip 302 relative to an auditory component. In this embodiment, a cap 242 is sized to fit over an end of the transducer 140. The end of the cap (not shown) is open to permit the movable tip 302 to extend therethrough. Alternately, the tip 302 may be attached to the end of the cap 242. The cap member 242 may be affixed to the transducer 140 in any appropriate manner including, without limitation, in a snap-fit arrangement, by welding and/or by adherence. Interconnected to opposing outside surfaces of the cap member 242 are first and second guide wires 244, 246. These guide wires 244, 246 extend toward and beyond the vibratory actuator tip 302 of the transducer 140. These guide wires 244, 246 are spaced to engage opposing surfaces of an ossicle bone such as the incus 122. In the present embodiment, the guide wires 244, 246 are bent to conform to the outside surface of the incus 122. It will be appreciated that these guide wires 244, 246 may be bent by a surgeon during the implant procedure in order to conform to an ossicular component. The wires may be provided to be non-compliant or compliant after bending (e.g., to apply a spring-loaded force in the event of post-implantation auditory component movement).

As in the guide embodiment of FIGS. 3-5, it will be appreciated that the guide wires 244, 246 may maintain or realign the ossicles to the transducer tip 302. In both embodiments of FIGS. 3-6 it will be noted that the guide armatures 202, 204 and guide wires 244, 246 do not clamp or otherwise extend around the auditory component. That is, while being disposed on opposing sides of the auditory component, the guides do not extend around the component such that the transducer 140 is mounted thereto. This may facilitate removal of the transducer 140.

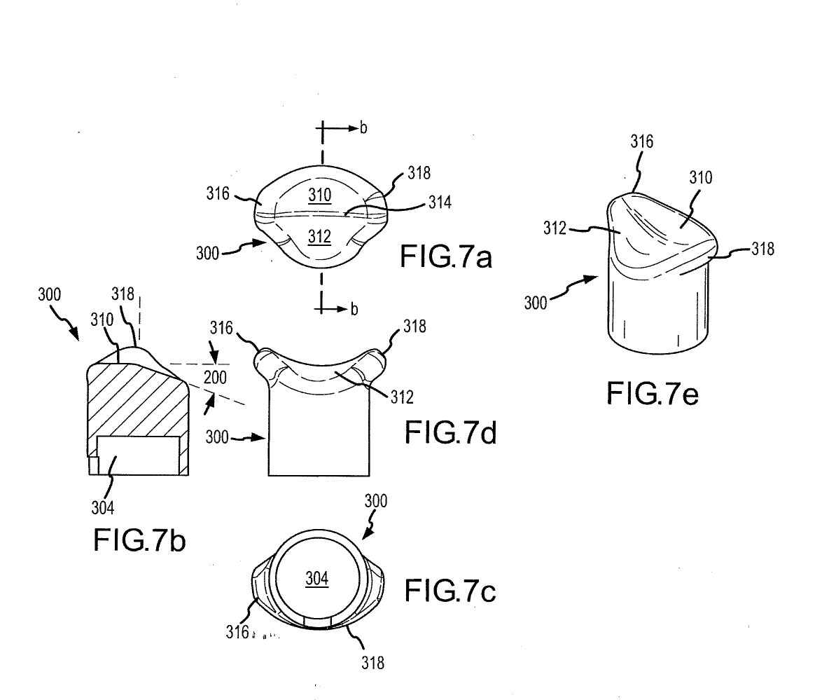

FIGS. 7-16 illustrate further embodiments of force loading connection apparatuses that provide centering for a transducer to ossicle interface. As illustrated in FIGS. 7-10, the force loading connection apparatus is formed as a connecting tip 300 that is adapted to fit over the end of the transducer vibratory actuator tip 302. In this regard, a rearward end of the connecting tip 300 contains an aperture 304 that is sized to conformably receive the tip 302. The connecting tip 300 may be interconnected to the tip 302 in any appropriate manner. For instance, the connecting tip may be crimped, adhered and/or welded to the transducer tip. A forward end of the connecting tip 300 has first and second concave surfaces 310, 312, wherein each of the concave surfaces 310, 312 have opposing surface portions that define an included angle therebetween (e.g., less than 180°) for restricting lateral movement of an auditory component positioned therebetween. In the arrangement shown, the first and second concave surfaces 310, 312 are partial cylindrical surfaces that have intersecting nonaligned axes (e.g., the respective axes intersect a center axis of tip 300 at differing angles). As shown, in FIG. 7b, the first and second partially cylindrical surfaces are offset 200 relative to one another. However, it will be appreciated that other angular offsets are possible and within the scope of the present invention.

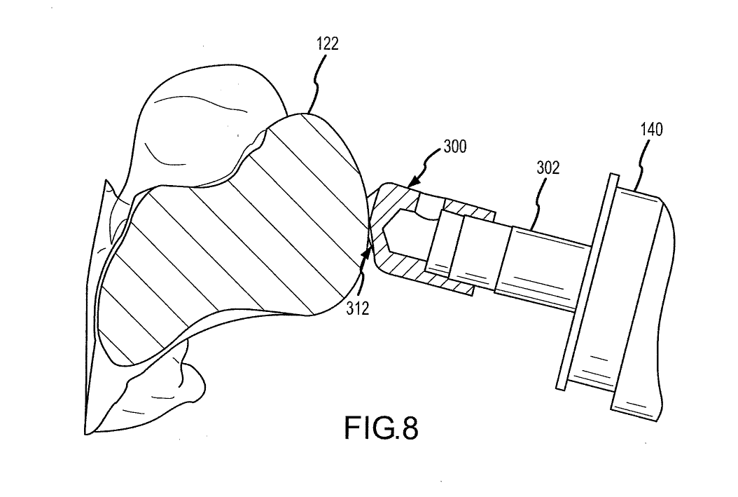

Utilization of the first and second offset concave surfaces 310, 312 permits selectively contacting an ossicle bone at different angular orientations. That is, utilization of the two surfaces 310, 312 permits flexibility in the positioning of the vibratory actuator transducer tip 302 (e.g., via the connecting tip 300) relative to a patient's ossicle bone such as the incus 112. See. FIG. 8. That is, the ossicle bone may contact the surface 310 or 312 best aligned with a contact area on the bone to optimize a centering effect. Further, an interface line 314 extending between the first and second concave surfaces 310, 312 may be textured to provide a degree of friction yet remove any sharp edges.

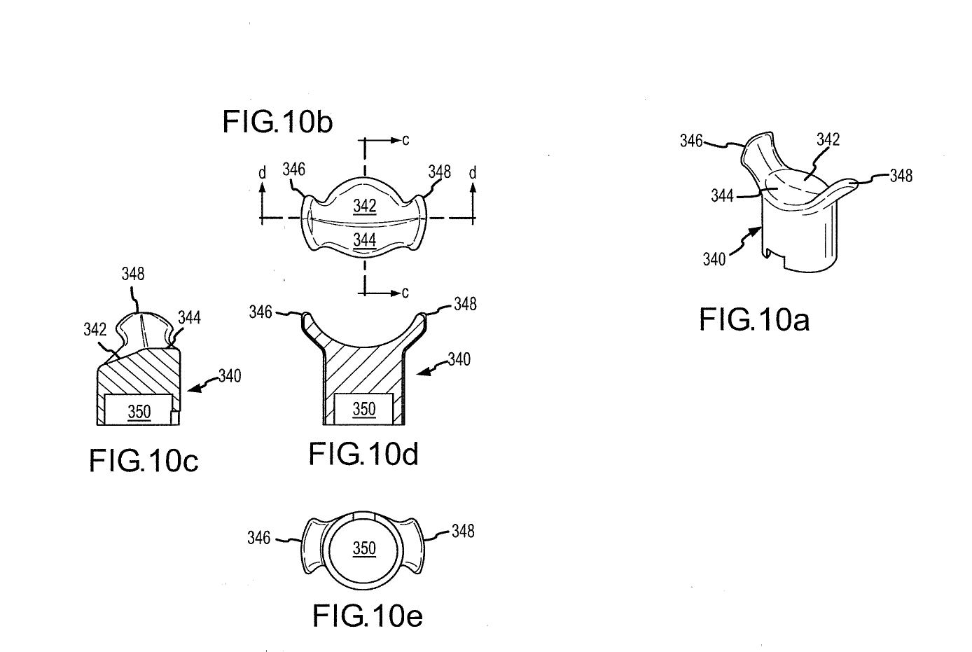

To further allow the connecting tip 300 to provide centering for the transducer to ossicle interface, the first and second surface 310, 312 may extend beyond the outside perimeter of a main body portion of the connecting tip 300. For instance, in reference to FIGS. 7a and 7d, it will be noted that the first and second surfaces 310, 312 extend to form first and second wings 316, 318. Collectively, these wings 316, 318 and the first and second surfaces 310, 312 form what may be defined as a ‘saddle’ into which the ossicular bone may be received. For instance, in reference to FIG. 10, it will be noted that the connecting tip 300 is adapted to receive a portion of the incus 112 within the saddle defined by the wings 316, 318. Accordingly, when disposed within the saddle, the incus 112 may be allowed to move laterally, or to rotate, but only to a limited degree relative to the contact axis defined by the transducer tip 302. That is, the saddle of the connecting tip 300 maintains the desired centered interface between the incus 112 and the transducer tip 302 while permitting some relative movement therebetween.

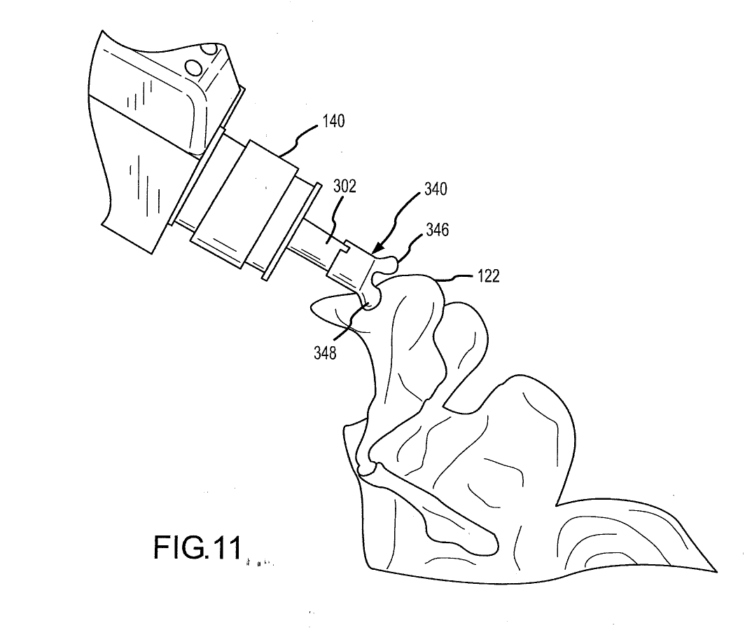

FIGS. 10a-10e and 11 show a further embodiment of a saddle-type connecting tip 340. Many of the features of the connecting tip 340 of FIGS. 10a-10e and 11 are common with the connecting tip 300 of FIGS. 7-9. For instance, the connecting tip 340 includes first and second concave surfaces 342, 344 and an aperture within its distal end 350 that is sized to receive a transducer tip. However, the first and second wings 346, 348 of the connecting tip 340 are more pronounced than those disclosed above. Accordingly, the resulting saddle formed by the first and second wings 346, 348 and first and second surfaces 342, 344 is deeper. This deep saddle may further limit the range of movement between the ossicle bone and the transducer vibratory actuator tip 302.

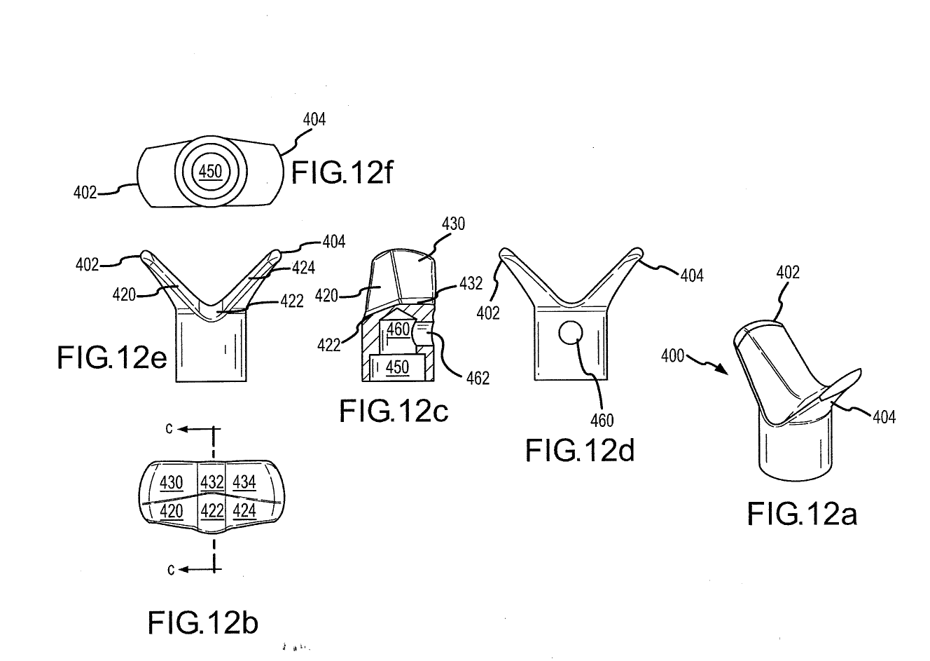

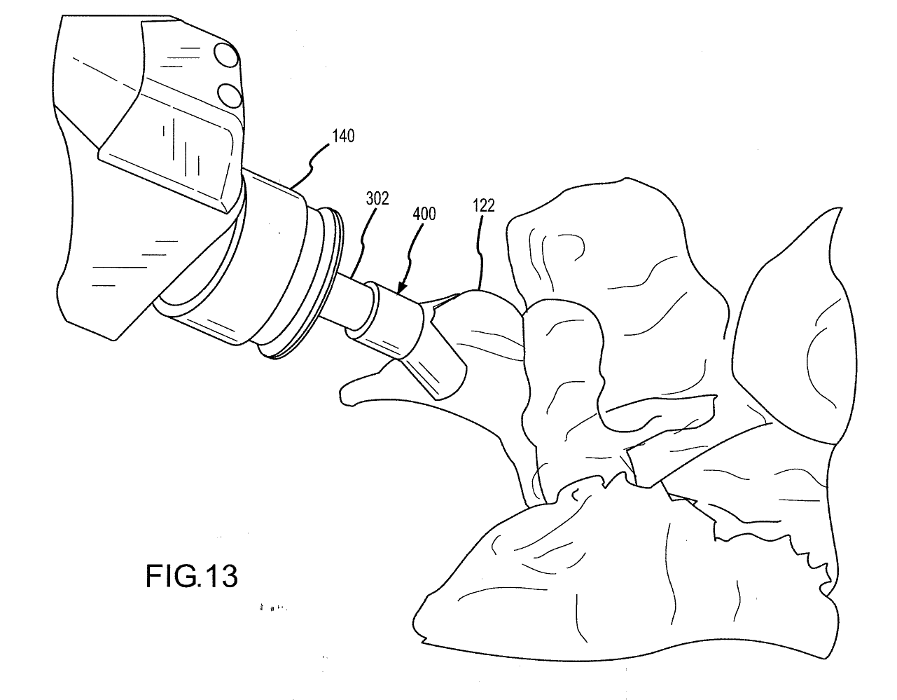

FIGS. 12-14 illustrate another embodiment of a force loading connection apparatus that provides centering for a transducer to ossicle interface. As shown in FIGS. 12a-12f, the apparatus is again formed as a connector tip 400 that is adapted for attachment to the transducer vibratory actuator tip 302 of the transducer 140. Again, the connector tip 400 includes an aperture in its rearward end that permits conformal attachment to a transducer tip via, for example, adherence, crimping and/or welding. As shown, the connector tip 400 includes first and second wings 402, 404 that are disposed at an angle relative to one another. As shown, the wings 402, 404 form a ‘V’ that is sized to receive a portion of an ossicle bone as illustrated in FIG. 13.

As shown, contact surfaces of the wings 402, 404 may, as with the saddle embodiments disclosed above, be aligned with different reference axes. Again, this may permit connecting the connecting tip 400 to an ossicle bone at different angular offsets. However, in contrast to the saddle embodiments disclosed above, the contact surfaces of the connecting tip 400 of FIGS. 12a-12f are non-continuous. That is, each contact surface includes three separate surfaces. For instance, a first set of contact surfaces 420, 422, 424 may each have a surface or curvature that is generally parallel to a common reference axis. Likewise, a second set of contact surfaces 430, 432, 434 may be generally parallel to a second reference axis. Accordingly, these reference axes may be disposed at an angle relative to one another and/or intersect. Again, an interface between the contact surfaces may be textured.

The connecting tip 400 may, as noted, include an aperture 450 for receiving a transducer vibratory actuator tip. Further, an additional portion of the interior of the connecting tip 400 may be removed for weight purposes. For instance, in reference to section AA of FIG. 10, it will be noted that adjacent to aperture 450, an interior portion 460 of the connecting tip 400 is removed for weight reduction purposes. A vent hole 462 extends through a sidewall of the connecting tip 400. This vent hole 462 may be utilized for out-gassing purposes when the connecting tip 400 is welded to a transducer tip, or to permit sterilant gas to enter the interior portion 460 during sterilization.

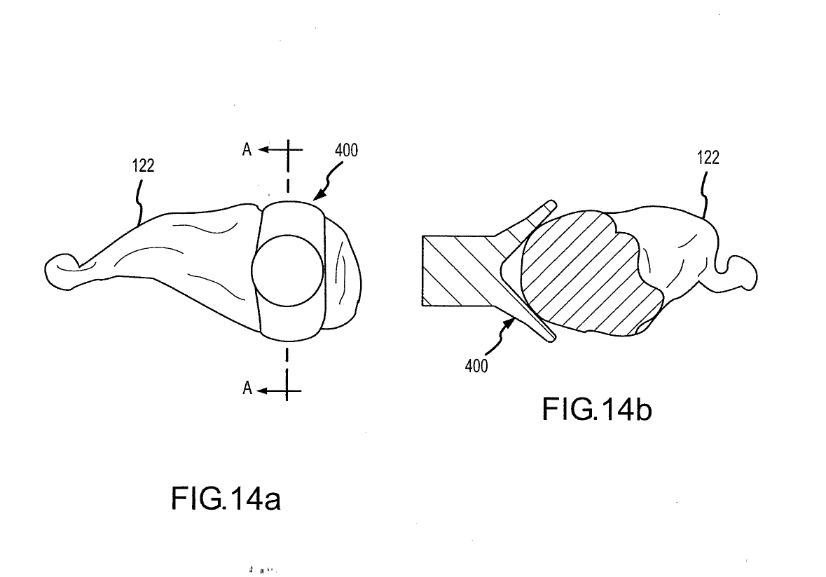

FIGS. 14a and 14b show the interconnection of the connecting tip 400 relative to an incus 122. As shown in FIG. 14b, the V-shaped connecting tip 400 provides, at a minimum, two points of contact between the contact surfaces 420-434 and the incus 122. Accordingly, due to the depth of the resulting V as well as the two contact points, the V-shaped connecting tip 400 may provide improved centering of the transducer tip 302 to an ossicle bone. As will be appreciated, this type of contact ensures that the two contact points will be reliably achieved despite variation among individuals in the size or curvature of the ossicle bone.

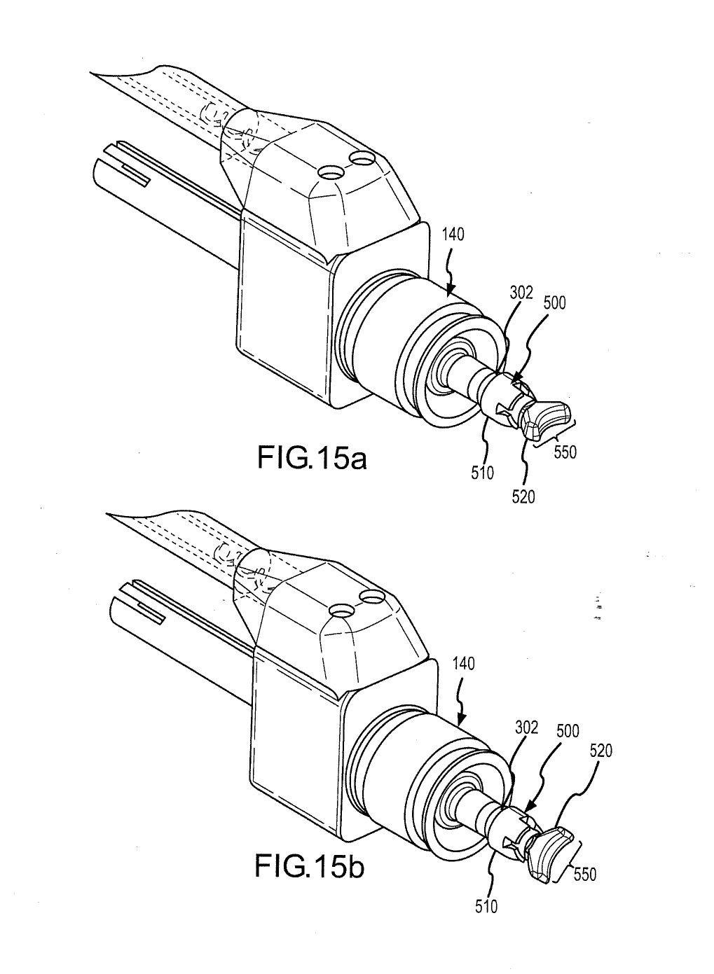

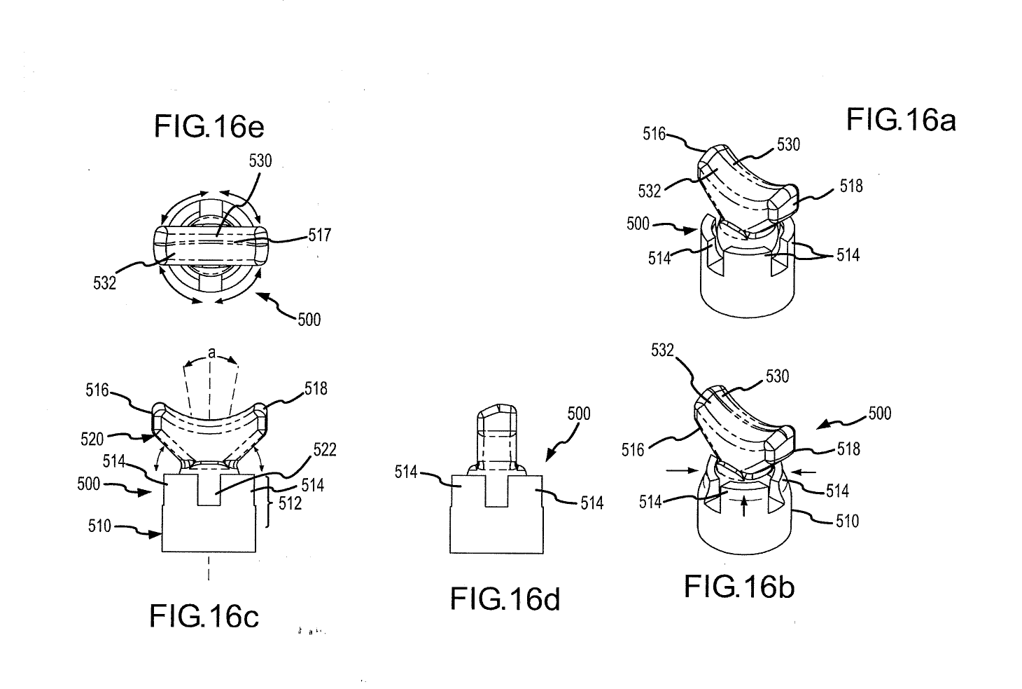

Referring now to FIGS. 15a-15e and FIGS. 16a-16e, a further embodiment of a force loading connection apparatus is illustrated which is similar to the embodiment shown in FIGS. 7a-7e. In particular, a force loading connection apparatus is formed as a connecting tip 500 that is adapted to fit over the end of the transducer vibratory actuator tip 302. In this arrangement, the connecting tip 500 may include a base portion 510 and a rotatable portion 520 interconnected thereto. More particularly, the rotatable portion 520 may comprise a rearward ball end 522 rotatably disposed within a forward cup-shaped end 512 of the base portion 510. For example, the base portion 510 may include a plurality of retention members 514 that may be bent in, or crimped, after placement of the ball end 522 of the rotatable portion 520 within the cup-shaped end 512 of the base portion 510 so as to capture the ball end 522 within the cup-shaped portion 512. In this regard, the ball end 522 may be captured while allowing for rotation and pivotable movement of the rotatable member 520 relative to the base portion 510 during positioning of the connecting tip 500 relative to an auditory component. That is, the rotatable portion 520 may rotate about a center axis of and pivot relative to the base portion 510. Relatedly, the interface between the ball end 522 and base portion 510 may be provided so that, upon contact engagement of rotatable portion 510 with an auditory component, relative movement between the base portion 510 and rotatable portion 520 may be restricted.

A distal, or forward end of the rotatable portion 520 is configured to have first and second concave surfaces 530, 532, respectively. Each of the surfaces 530, 532, have opposing surface portions that define an included angel therebetween for restricting auditory component movement. The concave surfaces 530, 532 may be partial cylindrical surfaces that have intersecting nonaligned axes, wherein a saddle is defined.

Utilization of the first and second offset concave surfaces 530, 532 permits contacting an ossicle bone at different angular orientations to optimize contact interface. For example, utilization of the two surfaces 530, 532 permits flexibility in the positioning of the transducer tip 302 relative to a patient's ossicle bone such as incus. That is, an ossicle bone may contact the surface 530 or 532 best aligned therewith. Further, an interface line 517 extending between the first and second concave surfaces 530, 532 can be rounded. For instance, the surface of the connector tip 500 may be bead blasted.

To further allow the connecting tip 500 to provide enhanced centering for the transducer to ossicle interface, the first and second surfaces 530, 532 may extend beyond the outside perimeter of a main body portion of the connecting tip 500. For instance, it will be noted that the first and second surfaces 530, 532 form first and second wings 516, 518. Collectively, these wings 516, 518 and the first and second surfaces 530, 532 define a saddle configuration into which an ossicular bone may be received. Accordingly, when disposed within the saddle, an incus 112 may be allowed to move laterally, or to rotate, to a limited degree relative to the axis defined by the transducer tip 502. That is, the saddle of the connecting tip 500 maintains the interface between the incus 112 and the transducer tip 302 while permitting some relative movement therebetween.

Due to the rotatable interface between the rotatable portion 520 and base portion 510, the connecting tip 500 may assume a plurality of orientations in relation to an auditory component. For example, the rotatable portion 520 may be rotated about a center axis that passes through the ball end 522 (e.g., 360° rotation). Further, the rotatable portion 520 may be pivoted, or rotated, within a predetermined angular range of motion relative to the base portion 510. For example, in the illustrated embodiment, a center axis of the rotatable portion 520 may be pivoted across a predetermined angular range a of about 30° relative to the base portion 520, e.g., the center axis may be pivoted about +15° relative to a center axis at the base portion 510.

As may be appreciated the rotatable portion 520 defines an opening 550 at a distal end thereof (e.g., for receipt of an auditory component therein). In turn, the rotatable and pivotable functionality of the rotatable portion 520 allows the opening 550 to be selectively oriented in at least two dimensions to facilitate contact engagement with an auditory member in a spectrum of different, selectable positions.

In all of the above noted embodiments, the force loading connection apparatuses permit the removal of the transducer 108 after initial implantation. That is, as no direct connection exists between the force loading connection apparatuses and the ossicle bone, the transducer may simply be retracted from the ossicle bone. As will be appreciated, this may facilitate removal and repair of transducers as well as replacement of transducers with next generation transducers. Furthermore, the force loading connection apparatuses provide the advantage of removability of the transducer 140, while reducing the potential for damage to the ossicies 120.

In a modified arrangement, rotatable portion 520 of connecting tip 500 may be spring-loaded to apply a spring force against an auditory component upon initial positioning. For example, a spring member may be interposed between the rotatable portion 520 and base portion 510.

In yet a further embodiment, a bendable distal tip member may be provided that is interconnected or interconnectable to a distal end of a vibratory actuator, wherein the distal member includes a center member (e.g., a wire member) and at least two outer guide members (e.g., wire members) that extend distally from a bendable portion. For example, the outer guide members may extend beyond a center member, wherein the bendable portion may be oriented (e.g., twisted and/or pivoted) to position an opening defined between the guide members as desired relative to an auditory component. Then, the distal member may be advanced to compressively contact the center member with an auditory component with the guide members located on opposing sides of the component for post-implantation centering. For example, the guide members may be rigid or compliant to provide a spring-force in response to post-implantation auditory component movement.

The above noted embodiments are provided for purposes of illustration. Numerous modifications, adaptations and extensions are contemplated and are intended to be within the scope of the present invention.

What is claimed is:

1. An apparatus for use with an implantable hearing aid transducer having a vibratory actuator for contacting an auditory component, comprising:
a centering device for maintaining a centered contact relationship between a distal end surface of a vibratory actuator of an implantable hearing aid transducer and an auditory component.

2. An apparatus as recited in claim 1, wherein said centering device comprises at least two guide members, that extend in a direction defined by at least a portion of the vibratory actuator.

3. An apparatus as recited in claim 2, wherein said at least two guide members of said centering device, as interconnected to said implantable hearing aid transducer, each extend beyond a distal end surface of said vibratory actuator.

4. An apparatus as recited in claim 3, wherein each of said at least two guide members comprises a portion that extends away from a center axis of said vibratory actuator.

5. An apparatus as recited in claim 3, wherein each of said at least two guide members comprise a portion that is at least one of bendable and deflectable away from a center axis of said vibratory actuator to apply a spring force against an auditory component in use.

6. An apparatus as recited in claim 3, wherein said at least two guide members are one of interconnected and adapted for interconnection to said implantable hearing aid transducer.

7. An apparatus as recited in claim 3, wherein said centering device comprises:
opposing first and second guide wires interconnected to a member that is one of affixed and affixable to an implantable hearing aid transducer.

8. An apparatus as recited in claim 3, wherein said centering device comprises:
opposing first and second armatures interconnected to a member that is one of affixed and affixable to said hearing aid transducer wherein distal ends of each of the first and second armatures extend outwardly away from one another.

9. An apparatus as recited in claim 3, wherein said at least two guide members are one of interconnected and interconnectable to a distal end of said vibratory actuator.

10. An apparatus as recited in claim 9, wherein said at least two guide members are defined by:
a tip member having at least two surface portions that define said at least two guide members and an included angle therebetween; and
wherein said tip member defines said distal end surface.

11. An apparatus as recited in claim 9, wherein said tip member comprises:
adjoining first and second concave surfaces, wherein said at least two guide members and the adjoining first and second concave surfaces define a saddle configuration.

12. An apparatus as recited in claim 3, wherein said at least two guide members define an opening therebetween, and wherein said centering device is adapted so that an orientation of said opening may be selectively adjusted relative to a center axis of said vibratory actuator.

13. An apparatus as recited in claim 12, wherein said opening comprises a center axis and said at least two guide members are positionable so that said opening center axis is selectively adjustable in at least one dimension relative to the center axis of said vibratory actuator.

14. An apparatus as recited in claim 12, wherein said opening comprises a center axis and said at least two guide members are positionable so that said opening center axis is selectively adjustable in two dimensions relative to the center axis of said vibratory actuator.

15. An apparatus as recited in claim 12, wherein said at least two guide members are rotatable about a center axis of said opening.

16. An apparatus as recited in claim 12, wherein said at least two guide members are at least one of pivotable together and rotatable together relative to a distal end of said vibratory actuator.

17. An apparatus as recited in claim 12, wherein said at least two guide members are both pivotable and rotatable relative to a distal end of said vibratory actuator.

18. An apparatus as recited in claim 12, wherein said at least two guide members extend from a member that is rotably interconnected to a distal end of said vibratory actuator.

19. An apparatus as recited in claim 12, wherein said at least two guide members are interconnected to a bendable member interconnected to a distal end of said vibratory actuator.

20. A method for use in the mechanical stimulation of an auditory component by an implantable hearing aid transducer, comprising:
contacting a distal end of a vibratory actuator of an implantable hearing aid transducer with an auditory component, wherein the vibratory actuator is displaceable in response to operation of the implantable hearing aid transducer;
positioning at least two guide members on opposing sides of the auditory component, wherein the at least two guide members are supportably interconnected to the implantable hearing aid transducer; and,
maintaining a desired centered relationship between the distal end of the vibratory actuator and the auditory component post-implantation by engaging at least one of the at least two guide members with a lateral aspect of the auditory component.

21. A method as recited in claim 20, wherein said maintaining step includes at least one of:
restricting post-implantation auditory component movement by said engagement; and,
applying a spring-loaded force by said at least one of the at least two guide members.

22. A method as recited in claim 20, wherein said contacting step includes:
selecting one of a plurality of differently-shaped contact surfaces provided at the distal end of the vibratory actuator for contact with said auditory component; and,
advancing said selected one of the plurality of differently-shaped contact surfaces relative to the auditory component into compressive engagement.
